(19) United States
(12) Patent Application Publication
(10) Pub. No.: US 2008/0293947 A1
(43) Pub. Date: Nov. 27, 2008

(54) Method of Preparation for Imidazolepyridines

(76) Inventors: Robert E. Halvachs, Belleville, IL (US); Gary L. Cantrell, Troy, IL (US); Peter X. Wang, Chesterfield, MO (US)

Correspondence Address: Mallinckrodt Inc., 675 McDonnell Boulevard, HAZELWOOD, MO, 63042, US

(21) Appl. No.: 12/094,643

(22) PCT Filed: Nov. 3, 2006
(86) PCT No.: PCT/US06/43156

§ 371 Date: May 22, 2008

Related U.S. Application Data
(60) Provisional application No. 60/740,058, filed on Nov. 28, 2005.

(51) Int. Cl. C07D 471/04

(52) U.S. Cl. 546/121

(57) ABSTRACT

The present invention relates to an improved process for preparing imidazo[1,2-a]pyridine-3-acetamides and more particularly, 6-methyl-2-p-tolylH-imidazo[1,2-a]pyridine.

### FIELD OF THE INVENTION

The present invention relates to an improved process for preparing imidazo[1,2-a]pyridine-3-acetamides and more particularly, 6-methyl-2-p-tolylH-imidazo[1,2-a]pyridine.

### BACKGROUND OF THE INVENTION

Imidazo[1,2-a]pyridine-3-acetamides are described extensively in the literature and contain the well known pharmaceutical zolpidem, N,N-dimethyl-2-[6-methyl-2-(4-methylphenyl)imidazo[1,2-a]pyridine-3-yl]acetamide, which has the following structural formula:

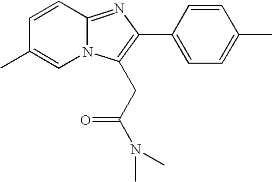

Zolpidem possesses anxiolytic, sedative, and hypnotic properties and is U.S. F.D.A. approved for short-term treatment of insomnia.

Among the problems associated with previous processes incorporating Zolpidem, the synthesis of the compound typically suffered from low yields due in part to the isolation and purification of the strong irritant, α-bromo-4-methylacetophenone.

Almost all previously described methods of synthesis have proceeded through the initial formation of the required imidazo[1,2-a]pyridine followed by the attachment of a suitable derivative on the 3-position and subsequent conversion to the desired acetamide derivative. One example, U.S. Pat. No. 4,794,185, describes a method of formation of compound (I), see below, via reaction of the aldehyde prepared in situ by acid hydrolysis from N,N-dimethyl-2,2-dimethoxyacetamide, isolation of the 3-substituted derivative, conversion of the hydroxyl group to the chloride with thionyl chloride and subsequent reduction of the chloro derivative to the imidazo[1,2-a]pyridine-3-N,N-dialkylacetamide derivative with sodium borohydride. This process suffers from the fact that it is difficult to obtain a suitable hydrolysis product of N,N-dimethyl-2,2-dimethoxyacetamide in situ and thus the reaction can not be taken to completion. Also the procedure is laborious and usually results in low overall yields.

Another example, EP 50,563 describes a process in which 6-methyl-2-(4-methylphenyl)-imidazo[1,2-a]pyridine is reacted to form 3-(N,N-dimethylaminoethyl)-6-methyl-2-(4-methylphenyl)-imidazo[1,2-a]pyridine. This compound is then treated with methyl iodide and subsequent derivatives are displaced with cyanide. The resulting cyano compound can then be converted to the desired derivative in several steps. Again this is a very laborious procedure producing low overall yields and utilizing toxic reagents.

Thus, prior methods of preparation of compound (I) require many steps, occur in low yield, use toxic reagents and involve complex procedures. Therefore, there is a need for a more economic and simpler commercial synthesis.

### SUMMARY OF THE INVENTION

Among the various aspects of the present invention is an improved process for preparing imidazo[1,2-a]pyridine-3-acetamides of structural formula (I) in general, and more particularly 6-methyl-2-p-tolylH-imidazo[1,2-a]pyridine, a key intermediate in the synthesis of Zolpidem.

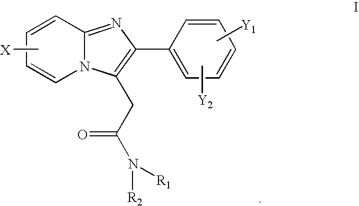

wherein:

X is a hydrogen or C₁₋₄ alkyl;

Y₁ and Y₂ are independently hydrogen or C₁₋₄ alkyl; and

R₁ and R₂ are independently methyl or C₁₋₄ alkyl.

In one embodiment, the invention comprises a process for the production of a substituted imidazolepyridine comprising selective bromination of a substituted acetophenone to form a brominated acetophenone; and reaction of the brominated acetophenone in mild basic solution with a substituted 2-aminopyridine to form the substituted imidazolepyridine.

In a further embodiment, the invention comprises a process for the production of imidazo[1,2-a]pyridine-3-acetamides such as N,N-dimethyl-2-[6-methyl-2-(4-methylphenyl)imidazo[1,2-a]pyridine-3-yl]acetamide (zolpidem). The process of this invention gives overall higher yields of zolpidem as compared to conventional processes by eliminating the isolation and purification of the strong irritant, α-bromo-4-methyl-acetophenone, since it is prepared in situ, transferred in solution and chemically transformed on addition to a reactive solution of the 2-amino-5-picoline. The result is savings in time, equipment, labor, transfer and yield losses.

In yet another embodiment of the present invention the production of a substituted imidazolepyridine comprises selective chlorination of a substituted acetophenone to form a chlorinated acetophenone; and reaction of the chlorinated acetophenone in mild basic solution with a substituted 2-aminopyridine to form the substituted imidazolepyridine. Optionally, bromide or iodide anions react with the chlorinated acetophenone to form the more reactive bromo or iodo analog in situ on replacement of the chloride. An example of this type of displacement is given in Rheinboldt, H. and Perrier, M.; JACS (1947) 69, 3148-9, which is incorporated herein by reference.

Other objects and aspects of the invention will be, in part, pointed out and, in part, apparent hereinafter.

### DETAILED DESCRIPTION OF THE PREFERRED EMBODIMENT(S)

The present invention provides an improved method for preparing imidazo[1,2-a]pyridine-3-acetamides, and more particularly the key intermediate in the synthesis of zolpidem, 6-methyl-N,N-dimethyl-2-p-tolylH-imidazo[1,2-a]pyridine. The general process comprising the selective halogenation of a substituted acetophenone is shown in reaction scheme 1.

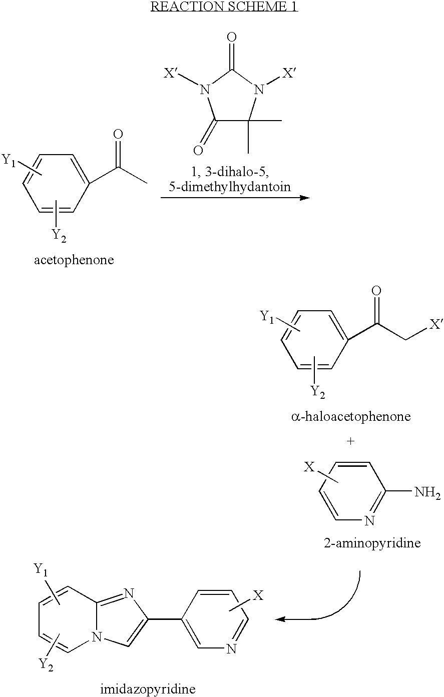

In one embodiment, the selective halogenation is selective bromination, shown in reaction scheme 2, and is as follows:

4′-methylacetophenone is brominated using the mild and efficient agent, 1,3-N,N-dibromo-5,5-dimethylhydantoin, giving α-bromo-4′-methylacetophenone also known as p-methylphenylacylbromide in excellent yield with minimal unreacted and over-brominated by-products. It was assessed to be superior to other brominating agents such as quaternary perbromides, N-bromosuccinimide, N-bromo-acetamide and bromine that have been reported in the preparation of α-bromoacetophenones. Solvents useful in the bromination may be comprised of but not limited to an organic liquid or mixtures of the following: chloroform, dichloromethane, fluorobenzene, chlorobenzene, methanol, ethanol, acetonitrile, and THF.

The strong acid catalyst that is present with the substituted acetophenone in the chosen solvent or mixture of solvents is selected from but not limited to concentrated sulfuric, hydrogen bromide, hydrogen chloride, strong organic acids such as methanesulfonic acid, benzenesulfonic acid, p-toluenesulfonic acid, trifluoromethanesulfonic acid and trifluoroacetic acid.

The subsequent condensation reaction requires an excess of a mild base such as alkali salts of carbonate, bicarbonate, di- and tri-phosphates, BICINE, TRICINE, TRIS, CAPS, CAPSO, EPPS, HEPES, MES, MOPS, PIPES, TAPS, TES, pyridine, triethylamine, diisopropylethylamine, N-methylmorpholine, N,N-dimethyl-aminopyridine, and mixtures thereof. 2-Amino substituted pyridines like 2-amino-5-picoline react with the α-bromoketone to condense in the presence of the selected base to form the imidazolepyridine ring system in high overall yield.

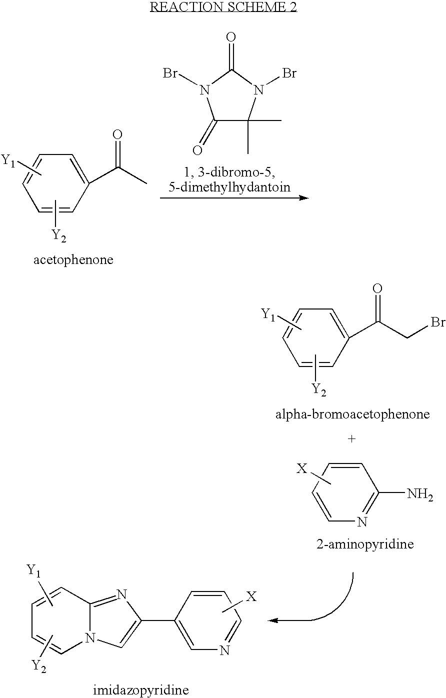

wherein X, Y₁ and Y₂ are independently hydrogen or C₁₋₄ alkyl.

In another embodiment, the selective halogenation is selective chlorination, shown in reaction scheme 3, and is as follows:

4′-methylacetophenone is chlorinated using the mild and efficient agent, 1,3-N,N-dichloro-5,5-dimethylhydantoin, giving α-chloro-4′-methylacetophenone also known as p-methylphenylacylchloride in excellent yield with minimal unreacted and over-chlorinated by-products. Solvents useful in the chlorination may be comprised of but not limited to an organic liquid or mixtures of the following: chloroform, dichloromethane, fluorobenzene, chlorobenzene, methanol ethanol, acetonitrile, and THF.

The strong acid catalyst that is present with the substituted acetophenone in the chosen solvent or mixture of solvents is selected from but not limited to concentrated sulfuric, hydrogen bromide, hydrogen chloride, strong organic acids such as methanesulfonic acid, benzenesulfonic acid, p-toluenesulfonic acid, trifluoromethanesulfonic acid and trifluoroacetic acid.

The subsequent condensation reaction requires an excess of a mild base such as alkali salts of carbonate, bicarbonate, di- and tri-phosphates, BICINE, TRICINE, TRIS, CAPS, CAPSO, EPPS, HEPES, MES, MOPS, PIPES, TAPS, TES, pyridine, triethylamine, diisopropylethylamine, N-methylmorpholine, N,N-dimethyl-aminopyridine, and mixtures thereof. 2-Amino substituted pyridines like 2-amino-5-picoline react with the α-chloroketone to condense in the presence of the selected base to form the imidazolepyridine ring system in high overall yield.

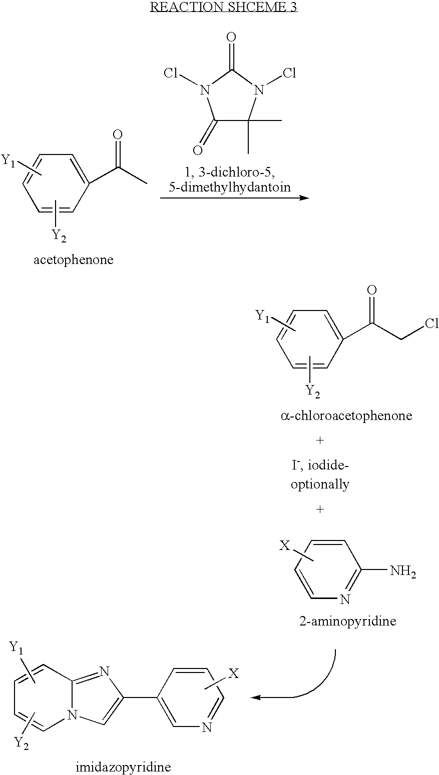

wherein X, Y₁ and Y₂ are independently hydrogen or C₁₋₄ alkyl.

In still another embodiment, the selective halogenation is selective iodination, shown in reaction scheme 4, and is as follows:

4′-methylacetophenone is iodinated using the mild and efficient agent, 1,3-N,N-diiodo-5,5-dimethylhydantoin, giving α-iodo-4′-methylacetophenone also known as p-methylphenylacyliodide in excellent yield with minimal unreacted and over-iodinated by-products. Solvents useful in the iodination may be comprised of but not limited to an organic liquid or mixtures of the following: chloroform, dichloromethane, fluorobenzene, chlorobenzene, methanol ethanol, acetonitrile, and THF.

The strong acid catalyst that is present with the substituted acetophenone in the chosen solvent or mixture of solvents is selected from but not limited to concentrated sulfuric, hydrogen bromide, hydrogen chloride, strong organic acids such as methanesulfonic acid, benzenesulfonic acid, p-toluenesulfonic acid, trifluoromethanesulfonic acid and trifluoroacetic acid.

The subsequent condensation reaction requires an excess of a mild base such as alkali salts of carbonate, bicarbonate, di- and tri-phosphates, BICINE, TRICINE, TRIS, CAPS, CAPSO, EPPS, HEPES, MES, MOPS, PIPES, TAPS, TES, pyridine, triethylamine, diisopropylethylamine, N-methylmorpholine, N,N-dimethyl-aminopyridine, and mixtures thereof. 2-Amino substituted pyridines like 2-amino-5-picoline react with the α-iodoketone to condense in the presence of the selected base to form the imidazolepyridine ring system in high overall yield.

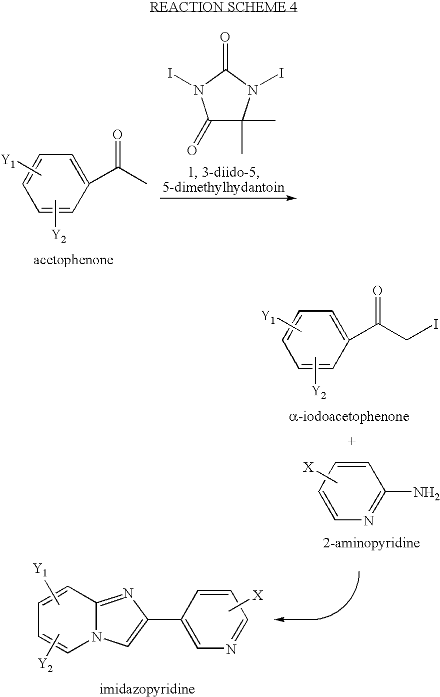

wherein X, Y₁ and Y₂ are independently hydrogen or C₁₋₄ alkyl.

In still another embodiment, the selective halogenation comprises a mixed halo hydantoin such as 1-bromo-3-chloro-5,5-dimethyl hydantoin, and is as follows:

4′-methylacetophenone is halogenated using the mild and efficient agent, 1-bromo-3-chloro-5,5-dimethyl hydantoin, giving a mixture of α-bromo-4′-methylacetophenone and α-chloro-4′-methylacetophenone in excellent yield with minimal unreacted and over-halogenated by-products. Solvents useful in the halogenation may be comprised of but not limited to an organic liquid or mixtures of the following: chloroform, dichloromethane, fluorobenzene, chlorobenzene, methanol ethanol, acetonitrile, and THF.

The strong acid catalyst that is present with the substituted acetophenone in the chosen solvent or mixture of solvents is selected from but not limited to concentrated sulfuric, hydrogen bromide, hydrogen chloride, strong organic acids such as methanesulfonic acid, benzenesulfonic acid, p-toluenesulfonic acid, trifluoromethanesulfonic acid and trifluoroacetic acid.

The subsequent condensation reaction requires an excess of a mild base such as alkali salts of carbonate, bicarbonate, di- and tri-phosphates, BICINE, TRICINE, TRIS, CAPS, CAPSO, EPPS, HEPES, MES, MOPS, PIPES, TAPS, TES, pyridine, triethylamine, diisopropylethylamine, N-methylmorpholine, N,N-dimethyl-aminopyridine, and mixtures thereof. 2-Amino substituted pyridines like 2-amino-5-picoline react with the α-bromoketone and α-chloroketone to condense in the presence of the selected base to form the imidazolepyridine ring system in high overall yield.

In a further embodiment, the invention comprises a process for the production of imidazo[1,2-a]pyridine-3-acetamides such as N,N-dimethyl-2-[6-methyl-2-(4-methylphenyl)imidazo[1,2-a]pyridine-3-yl]acetamide (zolpidem). The process of this invention gives overall higher yields of zolpidem as compared to conventional processes by eliminating the isolation and purification of the strong irritant, α-bromo-4-methyl-acetophenone, since it is prepared in situ, transferred in solution and chemically transformed on addition to a reactive solution of the 2-amino-5-picoline. The general process, shown in reaction scheme 5, wherein X, Y₁ and Y₂ are independently hydrogen or C₁₋₄ alkyl; and R₁ and R₂ are C₁₋₄ alkyl, is as follows:

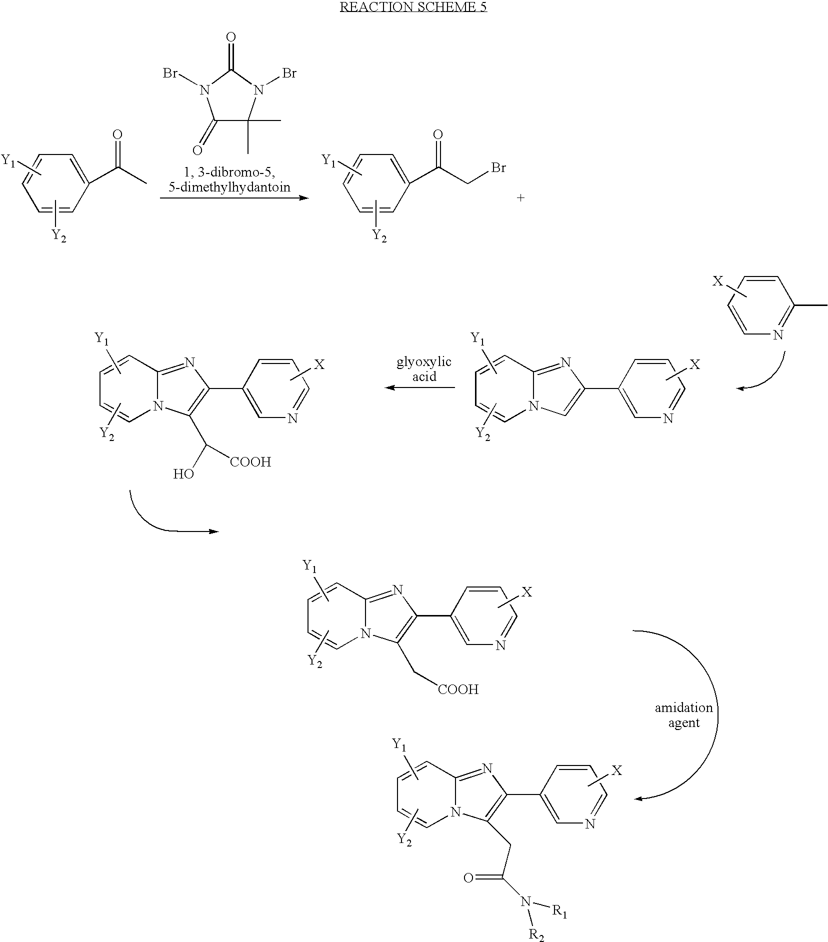

### EXAMPLES

The following non-limiting examples illustrate the invention.

### Example 1

4′-Methylacetophenone (402.6 g, 3 moles) and chloroform (1.6 L) was placed in a 3 L 3-necked flask fitted with a mechanical stirrer, a thermocouple connected to a heater controller, a condenser and a nitrogen sweep. The flask was initially placed in a water bath held at 40° C. Solid 1,3-N,N-dibromo-5,5-dimethylhydantoin (145.3 g, ˜0.5 mole) was added to the stirred solution followed by catalytic concentrated sulfuric acid (2.5 mL). The temperature rose to 45° C. Once the temperature had decreased to ˜40° C., the second portion of 1,3-N,N-dibromo-5,5-dimethylhydantoin (145.3 g, ˜0.5 mole) was added. Again, the temperature rose to 45° C. and then slowly cooled back to ˜40° C. whereupon the last portion of 1,3-N,N-dibromo-5,5-dimethylhydantoin (145.3 g, ˜0.5 mole) was added. A heating mantle was placed under the flask and the solution was held at 45° C. with stirring until the orange color dissipated. The overall addition reaction time was 2.5-3 hours. The HPLC analysis of the crude bromoketone solution showed 5-6% unreacted ketone, ˜2% dibrominated product and ˜92% α-bromo-4′-methylacetophenone. The solid, 5,5-dimethyl-hydantoin, was removed by filtration and washed with chloroform (˜200 mL). The chloroform filtrate containing the crude α-bromo-4′-methylacetophenone was placed in an addition funnel for transfer.

In a separate three-necked reaction flask fitted with a mechanical stirrer, thermocouple/controller condenser and heating mantle was added 2-amino-5-picoline (292 g's, 2.7 moles), chloroform (1 L) and sodium bicarbonate (192 g's). The crude α-bromoketone solution was added to this mixture with good stirring and CO₂ evolved. The mixture was heated to reflux at 60° C. for four hours. Water (1.2 L) was then added and heating was continued to reflux for 30 minutes. The stirring was stopped and the separate chloroform layer was taken off. Chloroform (100 mL) was added with stirring to the aqueous phase. The stirring was stopped and the chloroform phase was removed. The chloroform extracts were placed in a flask. Chloroform (˜1 L) was removed by simple distillation. t-Butylmethylether (2 L) was poured into the chloroform concentrate to facilitate precipitation. The stirred suspension was cooled to 5-10° C. The white solid was separated by vacuum filtration, washed with isopropyl alcohol and dried in an oven ˜60° C. The yield of 6-methyl-2-p-tolylH-imidazo[1,2-a]pyridine was ˜90% from the 2-amino-5-picoline.

### Example 2

4′-Methylacetophenone (134 g's, 1 mole) and chloroform (500 mL) is placed in a 1 L 3-necked flask fitted with a mechanical stirrer, a thermocouple connected to a heater controller, a condenser and a nitrogen sweep. The flask is initially placed in a water bath held at 40° C. Solid 1,3-N,N-dichloro-5,5-dimethylhydantoin (145.3 g's, ˜0.5 mole) is added to the stirred solution followed by the addition of catalytic concentrated sulfuric acid (0.75 mL). The temperature rises to 45° C. Once the temperature had decreased to ˜40° C., the second portion of 1,3-N,N-diichloro-5,5-dimethylhydantoin (50 g's, ˜0.0.2 mole) is added. Again, the temperature rises to 45° C. and then slowly cools back to ˜40° C.; whereupon, the last portion of 1,3-N,N-dichloro-5,5-dimethylhydantoin (50 g's, ˜0.2 mole) is added. A heating mantle is placed under the flask and the solution is held at 45° C. with stirring until the dark yellow color dissipates. The overall addition reaction time is 2.5-3 hours. The solid, 5,5-dimethyl-hydantoin, is removed by filtration and washed with chloroform (˜25 mL). The chloroform filtrate containing the crude α-chloro-4′-methylacetophenone is placed in an addition funnel for transfer.

In a separate three-necked reaction flask fitted with a mechanical stirrer, thermocouple/controller condenser and heating mantle is added 2-amino-5-picoline (100 g's, 0.9 moles), chloroform (350 mL) and sodium bicarbonate (65 g's). The crude α-chloroketone solution is added to this mixture with good stirring and CO₂ evolves. The mixture is heated to reflux at 60° C. for four hours. Water (400 mL) is then added and heating is continued to reflux for 30 minutes. The stirring is stopped and the separate chloroform layer is taken off. Chloroform (35 mL) is added with stirring to the aqueous phase. The stirring is stopped and the chloroform phase is removed. The chloroform extracts are placed in a flask. Chloroform (˜350 mL) is removed by simple distillation. t-Butylmethylether (650 mL) is poured into the chloroform concentrate to facilitate precipitation. The stirred suspension is cooled to 5-10° C. The white solid is separated by vacuum filtration, washed with isopropyl alcohol and dried in an oven ˜60° C. The yield of 6-methyl-2-p-tolylH-imidazo[1,2-a]pyridine is ˜90% from the 2-amino-5-picoline.

What is claimed is:

1. A process for the production of a substituted imidazolepyridine comprising selective halogenation of a substituted acetophenone to form a halogenated acetophenone; and reaction of the halogenated acetophenone in mild basic solution with a substituted 2-aminopyridine to form the substituted imidazolepyridine.

2. The process of claim 1 wherein the selective halogenation is selected from the group consisting of selective bromination, selective chlorination and selective iodination.

3. The process of claim 2 wherein the mild basic solution comprises a base selected from the group consisting of alkali salts of carbonate, bicarbonate, di- and tri-phosphates, BICINE, TRICINE, TRIS, CAPS, CAPSO, EPPS, HEPES, MES, MOPS, PIPES, TAPS, TES, pyridine, triethylamine, diisopropylethylamine, N-methylmorpholine, N,N-dimethyl-aminopyridine, and mixtures thereof.

4. The process of claim 2 wherein the substituted acetophenone is 4′-methyl-acetophenone.

5. The process of claim 4 wherein the substituted acetophenone is brominated using 1,3-N,N-dibromo-5,5-dimethylhydantoin.

6. The process of claim 4 wherein the substituted acetophenone is chlorinated using 1,3-N,N-dichloro-5,5-dimethylhydantoin.

7. The process of claim 4 wherein the substituted acetophenone is iodinated using 1,3-N,N-diiodo-5,5-dimethylhydantoin.

8. The process of claim 5 wherein the halogenated acetophenone is α-bromo-4-methylacetophenone.

9. The process of claim 6 wherein the halogenated acetophenone is α-chloro-4-methylacetophenone.

10. The process of claim 7 wherein the halogenated acetophenone is α-iodo-4-methylacetophenone.

11. The process of claim 10 wherein the α-iodo-4-methylacetophenone is prepared from the α-bromo- or α-chloro-4-methylacetophenone and a metal iodide.

12. The process of claim 11 wherein the metal iodide is selected from the group consisting of lithium iodide, sodium iodide, potassium iodide, cesium iodide, copper(I) iodide, zinc iodide, stannous iodide and iron iodide.

13. The process of claim 2 wherein the halogenation occurs in the presence of at least one organic solvent.

14. The process of claim 13 wherein the organic solvent is selected from the group consisting of chloroform, dichloromethane, fluorobenzene, chlorobenzene, methanol, ethanol, acetonitrile, and THF.

15. The process of claim 14 wherein the substituted 2-aminopyridine is 2-amino-5-picoline.

16. The process of claim 15 wherein the substituted imidazolepyridine is 6-methyl-2-p-tolylH-imidazo[1,2-a]pyridine.

17. A process for the production of a compound or salt thereof of the formula 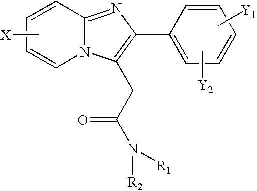
wherein:
X is hydrogen or C₁₋₄ alkyl;
Y₁ and Y₂ are independently hydrogen or C₁₋₄ alkyl; and
R₁ and R₂ are independently methyl or C₁₋₄ alkyl;
the process comprising selective halogenation of a substituted acetophenone to form a halogenated acetophenone; reaction of the halogenated acetophenone in mild basic solution with a substituted 2-aminopyridine to form a substituted imidazolepyridine; and
reacting the substituted imidazolepyridine with a hydrogenolysis agent followed by reaction with an amidation agent to produce an imidazo[1,2-a]pyridine-3-acetamide.

18. The process of claim 17 wherein the selective halogenation is selected from the group consisting of selective bromination, selective chlorination, and selective iodination.

19. The process of claim 18 wherein the mild basic solution comprises a base selected from the group consisting of alkali salts of carbonate, bicarbonate, di- and tri-phosphates, BICINE, TRICINE, TRIS, CAPS, CAPSO, EPPS, HEPES, MES, MOPS, PIPES, TAPS, TES, pyridine, triethylamine, diisopropylethylamine, N-methylmorpholine, N,N-dimethyl-aminopyridine, and mixtures thereof.

20. The process of claim 18 wherein the substituted acetophenone is 4′-methyl-acetophenone.

21. The process of claim 20 wherein the substituted acetophenone is brominated using 1,3-N,N-dibromo-5,5-dimethylhydantoin.

22. The process of claim 20 wherein the substituted acetophenone is chlorinated using 1,3-N,N-dichloro-5,5-dimethylhydantoin.

23. The process of claim 20 wherein the substituted acetophenone is iodinated using 1,3-N,N-diiodo-5,5-dimethylhydantoin.

24. The process of claim 21 wherein the halogenated acetophenone is α-bromo-4-methylacetophenone.

25. The process of claim 22 wherein the halogenated acetophenone is α-chloro-4-methylacetophenone.

26. The process of claim 23 wherein the halogenated acetophenone is α-iodo-4-methylacetophenone.

27. The process of claim 26 wherein the α-iodo-4-methylacetophenone is prepared from the α-bromo- or α-chloro-4-methylacetophenone and a metal iodide.

28. The process of claim 27 wherein the metal iodide is selected from the group consisting of lithium iodide, sodium iodide, potassium iodide, cesium iodide, copper(I) iodide, zinc iodide, stannous iodide and iron iodide.

29. The process of claim 18 wherein the halogenation occurs in the presence of at least one organic solvent.

30. The process of claim 29 wherein the organic solvent is selected from the group consisting of chloroform, dichloromethane, fluorobenzene, chlorobenzene, methanol, ethanol, acetonitrile, and THF.

31. The process of claim 30 wherein the substituted 2-aminopyridine is 2-amino-5-picoline.

32. The process of claim 31 wherein the substituted imidazolepyridine is 6-methyl-2-p-tolylH-imidazo[1,2-a]pyridine.

33. The process of claim 18 wherein:
X, Y₂, R₁ and R₂ are methyl;
Y₁ is hydrogen; and
the imidazo[1,2-a]pyridine-3-acetamide is N,N-dimethyl-2-[6-methyl-2-(4-methylphenyl)imidazo[1,2-a]pyridine-3-yl]acetamide.
